(19) United States
(12) Patent Application Publication
(10) Pub. No.: US 2011/0288592 A1
(43) Pub. Date: Nov. 24, 2011

(54) Side Opening Bone Fastener System

(75) Inventor: Laurence M. McKinley, Escondido, CA (US)

(73) Assignee: AEOLIN LLC, Escondido, CA (US)

(21) Appl. No.: 13/074,580

(22) Filed: Mar. 29, 2011

Related U.S. Application Data
(60) Provisional application No. 61/319,190, filed on Mar. 30, 2010.

(51) Int. Cl. A61B 17/88; A61B 17/86

(52) U.S. Cl. 606/279; 606/305

(57) ABSTRACT

A bone fastener assembly and a method of stabilizing a bone using such an assembly are presented. The bone assembly includes a bone fastener and a connecting member for holding a stabilizing member, such as a rod or plate, wherein the connecting member has a side-opening for accepting the rod therein. Optionally the bone fastener system also permits the bone fastener to pivot relative to a longitudinal axis of the assembly, and is provided with an armature that allows the connecting member to be offset from the longitudinal axis of the bone fastener and for the connecting member to swivel about the longitudinal axis of the assembly. By allowing the rod to be inserted into the connecting member via this side-opening the bone fastener provides a system that is particularly useful for minimally invasive surgeries.

### CROSS-REFERENCE TO RELATED APPLICATIONS

The present application is claims priority to U.S. Provisional Patent No. 61/319,190, filed Mar. 30, 2010, the disclosure of which is incorporated herein by reference.

### FIELD OF THE INVENTION

The current invention is directed to a bone fastener system; and more specifically to a bone fastener system having a stabilizing member connector with a side-opening for accepting stabilizer rods.

### BACKGROUND OF THE INVENTION

A multitude of orthopedic and neurological procedures use fasteners of one kind or another to connect one bone, or bone fragment, to another. Among these the most common use is in the connection of one vertebra of the human spine to another vertebra. In particular, the vertebrae of the human spine are positioned in a delicate arrangement that evenly distributes the force between the adjacent vertebrae and allows the spine to flex and twist with remarkable freedom. However, this delicate arrangement can be easily disrupted by a number of external stresses such as age, trauma, disease or general malformation.

Most conventional fastener systems operate through a plurality of bone fasteners interconnected by stabilizing rods. Although the specific design of these fasteners and rods have taken a number of different forms, in the vast majority of these systems the rod is held by a rod engagement seat that has an opening at the top of the rod engagement seat. While this arrangement provides excellent support in helping fuse adjacent vertebrae in most cases, sometime a lateral or “side-entry” rod engagement seat is desired. Specifically, in minimally invasive surgery it is important to have fixation systems that provide the greatest possible number degrees of freedom. Having a system with the capability to allow side-entry of the rod enhances the surgeon's ability to provide a full-range of fixation geometries with minimal intrusion into the patient.

Accordingly, a need exists for an improved fastener system that allows for a lateral or side-entry of the rod in relation to the vertebral fastener itself.

### SUMMARY OF THE INVENTION

The current invention is directed to a bone fastener system incorporating a side-entry connector seat.

In one embodiment, the side-opening bone fastener system for connecting a bone to a stabilizing member includes:
bone fastener having an elongated shank including a bone engaging portion at a first end, a head at an opposite, second end, and a longitudinal screw axis extending therebetween; and
a connecting member interconnected with said bone fastener, said connecting member having a longitudinal connecting member axis extending in the direction of the longitudinal screw axis, and a rod receiving channel extending at least partially through the connecting member transverse to the longitudinal axis for receiving a spinal rod, the receiving channel having an opening formed in a lateral face of the connection member along the axis of the stabilizing member but laterally offset therefrom such that the stabilizing member may be placed into the receiving channel of the connecting member laterally from the side of said connecting member.

In another embodiment, the head is elongated in a direction transverse to the longitudinal screw axis such that the bone fastener is generally “T”-shaped.

In still another embodiment, the connecting member is rotatably mounted to the bone fastener such that the connecting member rotates about at least one of the longitudinal axis of the connecting member and an axis transverse to the longitudinal connecting member axis.

In yet another embodiment, the connecting member is rotatably mounted to the bone fastener such that the connecting member rotates about the longitudinal axis of the fastener in a plane horizontal to the longitudinal axis of the fastener.

In still yet another embodiment, the stabilizing member receiving channel extends fully through the connecting member.

In still yet another embodiment, the stabilizing member is an elongated rod.

In still yet another embodiment, the assembly is made of stainless steel.

In still yet another embodiment, the head of the bone fastener further comprises a recess disposed in the head thereof and designed to receive a screw driver from the group consisting of: flat-headed, phillips head, allen wrench, and square headed.

In still yet another embodiment, the screw further comprises a slotted groove disposed longitudinally along the tip and designed to allow the screw to be self-tapped.

In still yet another embodiment, the connecting member further comprising a threaded channel and a cooperatively threaded mating nut having a plurality of engaging teeth disposed on the lower surface thereof, said threaded channel and mating nut being disposed such that said nut frictionally locks the stabilizing member within the channel of the connecting member.

In still yet another embodiment, the connecting member further comprises a stabilizing member contact member disposed between the stabilizing member and the mating nut, such that the threaded mating nut urges a contact surface of the stabilizing member contact member against the stabilizing member to lock the stabilizing member within the channel of the connecting member. In one such embodiment, the contact surface of the stabilizing member contact member is shaped to conform to the outer contour of the stabilizing member. In another such embodiment, the contact surface of the stabilizing member contact member has at least one raised feature thereon to increase the frictional force exerted on the stabilizing member when the stabilizing member contact member is engaged thereagainst.

In still yet another embodiment, the stabilizing member contact member is disposed within the channel opposite the lateral opening in the connecting member. In one such embodiment, the stabilizing member contact member is an elongated U-shaped member, wherein the opening in the U-shaped member runs along an axis parallel to the lateral channel opening in the connecting member. In another such embodiment, the stabilizing member contact member further comprises a retaining member that securely interconnects the stabilizing member contact member to the connecting member. In still another such embodiment, the retaining member interconnects with the connecting member via a key-hole mechanism. In yet another such embodiment, the stabilizing member contact member has a contact surface for engaging the stabilizing member, and wherein said contact surface has at least one raised feature thereon to increase the frictional force exerted on the stabilizing member when the stabilizing member contact member is engaged thereagainst.

In still yet another embodiment, the system includes an offset linkage interconnecting said connecting member and the head of the bone fastener, wherein the connecting member is disposed on the offset linkage such that the longitudinal connecting member axis is laterally offset from the longitudinal screw axis by a predefined distance. In one such embodiment, the offset linkage is pivotally interconnected with said head such that the connecting member azimuthally pivots about the head of said bone fastener; and wherein the connecting member is rotatably mounted to the offset linkage such that the connecting member rotates about the longitudinal connecting member axis. In another such embodiment, the connecting member and the offset linkage are a single integrated piece. In still another such embodiment, the lateral offset distance between the longitudinal screw axis and longitudinal connecting member axis is fixed. In yet another such embodiment, the lateral offset distance between the longitudinal screw axis and longitudinal connecting member axis is variable. In still yet another such embodiment, the system comprises a plurality of interchangeably removable offset linkages each having a different predefined fixed offset distance.

The invention is also directed to a vertebral alignment/fixation method comprising utilizing a plurality of the side-opening bone fastener systems to align at least one vertebral body.

### BRIEF DESCRIPTION OF THE FIGURES

Various examples of the present invention will be discussed with reference to the appended drawings. These drawings depict only illustrative examples of the invention and are not to be considered limiting of its scope.

FIG. 1a is a perspective view of one embodiment of a bone fastener assembly according to the present invention;

FIG. 1b is a side view of the bone fastener shown in FIG. 1a;

FIG. 1c is a top view of the bone fastener shown in FIG. 1a;

FIG. 1d is an exploded view showing the interconnection between the rod connector and the fastener body of the bone fastener shown in FIG. 1a;

FIG. 2a shows a side view of a second embodiment of a bone fastener assembly according to the present invention;

FIG. 2b shows an exploded view showing the interconnecting between the rod connector and the fastener body of the bone fastener shown in FIG. 2a;

FIG. 3 shows a perspective view of a third embodiment of a bone fastener assembly according to the present invention;

FIG. 3b is a side view of the bone fastener shown in FIG. 3a;

FIG. 3c is a top view of the bone fastener shown in FIG. 3a; and

FIG. 3d is an exploded view showing the interconnection between the rod connector and the fastener body of the bone fastener shown in FIG. 3a.

### DETAILED DESCRIPTION OF THE INVENTION

Embodiments of a bone fastener assembly according to the present invention include a bone fastener and a connecting member for holding a stabilizing member, such as a rod or plate that allows the stabilizing member to be inserted into to connecting member through a side opening that is not located atop the longitudinal axis of the bone fastener. In an optional embodiment, the connecting member may be offset and may also independently swivel about the longitudinal axis of the assembly. The combination of such an offset with the side or lateral opening arrangement of the connecting member provides the bone fastener assembly an unprecedented degree of freedom in positioning an interconnecting rod to that assembly.

### Definitions

Bone Fastener: For the purposes of this invention, a bone fastener is defined as any device suitable for engaging a bone, including, a screw, pin, nail, bolt, staple, hook, etc.

Stabilizing Member: For the purposes of this invention, a stabilizing member is defined as any device suitable for interconnecting and stabilizing bone, including, plates, rods, etc.

Connecting Member: For the purposes of this invention, a connecting member may include any suitable member for assembling a construct for stabilizing bones of a patient such as a plate engaging stud, rod holder, etc.

### Description

The bone fastener assembly of the current invention is comprised of three fundamental parts: a bone fastener for anchoring the assembly to a bone, a rotatable connecting member for holding a stabilizing member such as a plate rod, etc., and the stabilizing member itself. The following description details exemplary embodiments of these bone fastener assembly of the current invention.

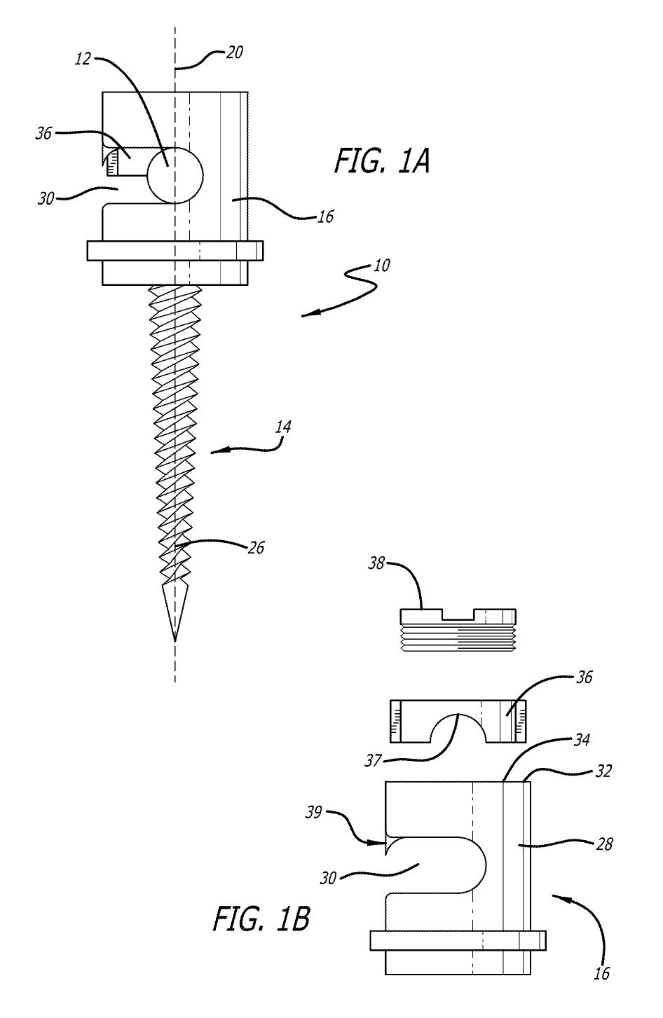

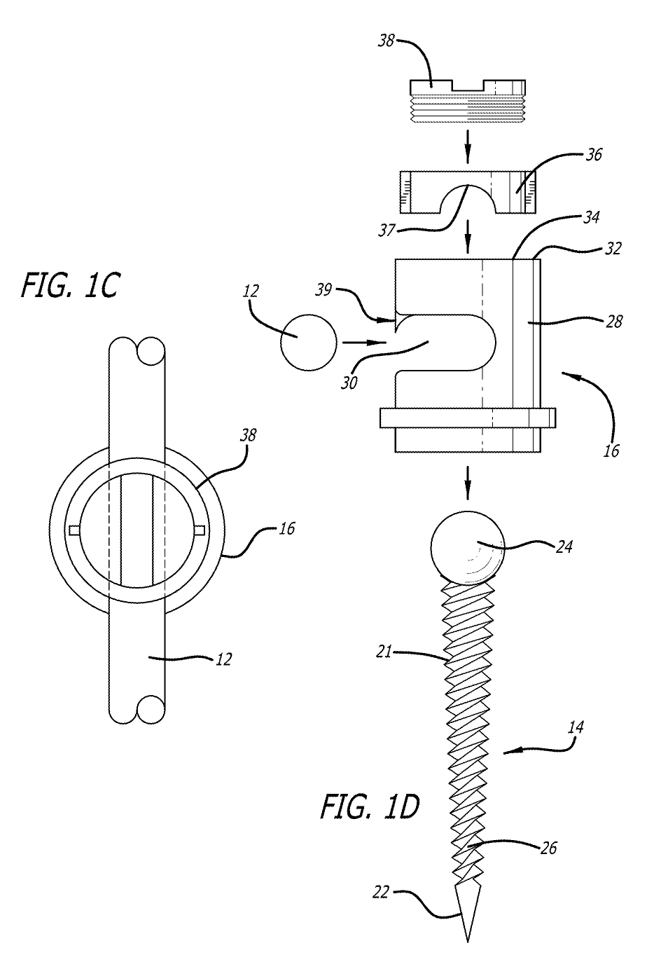

FIGS. 1a to 1d illustrate a bone fastener assembly in the form of a pedicle screw assembly 10 coupled to a spinal rod 12. As shown, each pedicle screw assembly 10 includes a bone fastener in the form of a screw 14 having a “T” shaped head, and a connecting member in the form of a rod holder 16, having an opening 18 for accepting the rod that is disposed in the side, i.e., on a lateral face, of the connecting member. The assembly includes a longitudinal axis 20 through the rod holder 16 and screw 14.

First, with regard to the exemplary embodiment shown in FIGS. 1a to 1d, in this first embodiment the connecting member is principally designed to allow a rod, stud, etc. to be secured to the bone fastener via a lateral or side opening. In the embodiment shown in FIG. 1, the connecting member 16 includes a generally cylindrical body 28 having a transverse passageway 30 extending through the body for receiving a rod, stud, etc. 12. In this invention, the transverse passageway 30 is open in a lateral face of the connection member 16 and in the direction of the axis of the rod 12, but laterally offset therefrom to allow the rod to be placed into the connecting member 16 with a single transverse motion from the side of the connecting member.

To secure the rod within the passageway 30 any suitable securing means may be used. As shown in FIG. 1, in one embodiment a screw thread 32 spirals from an upper opening 34 distally into the cylindrical body 28. In the embodiment shown, the connecting member 16 is also provided with a rod contacting member 36 that slidably engages within the upper opening 34 for engaging the stabilizing means, in this case the rod. The rod holder contacting member 36 preferably is provided with convex/spherical surface 37 to match the shape of the rod. In addition, in a preferred embodiment, the upper opening 34 and the rod holder contacting member 36 are provided with cooperative structures such that the rotational orientation of the rod holder contacting member 36 is controlled. A locking nut 38 engages the thread 32 in the upper opening of the cylindrical body 28 of the connecting member 16 to force the contacting member 36 down onto the rod 12. In the preferred embodiment shown in FIGS. 1a to 1d, and as best shown in FIGS. 1a and 1d, the mouth of the passageway 30 is also provided with an extended lip 39 designed to partially obscure the passageway 30 such that the rod 12, once positioned in the passageway will be held therein until the rod is locked down by the rod contact member 36 and lock nut 38.

As best seen in FIGS. 1a to 1c, the connecting member 16 may include a swivel connection, which allows for the connecting member to be rotated about the longitudinal axis 20 in a plane horizontal to the longitudinal axis 20 of the bone screw 10. This horizontal. swiveling allows the connecting member 16 to be moved to any desired position independent of the position of the screw 14 itself.

As discussed above, although any suitable bone fastener may be used, in one embodiment, shown in FIG. 1, the screw 14 includes an elongated shank 21 having a tip 22 at a distal end, a head 24 at a proximal end, and a longitudinal axis 20 extending therebetween. A thread 26 spirals around the shank such that the screw 14 may be threaded into a bone. As shown in FIG. 1, in a preferred embodiment the head 24 is a “T”-shape formed by the arrangement of a cylinder disposed transverse to the longitudinal axis 20 of the screw 14. However, it should be understood that any head shape may be used that allows the use of a connecting member having a side-opening for insertion of a rod in accordance with the current invention.

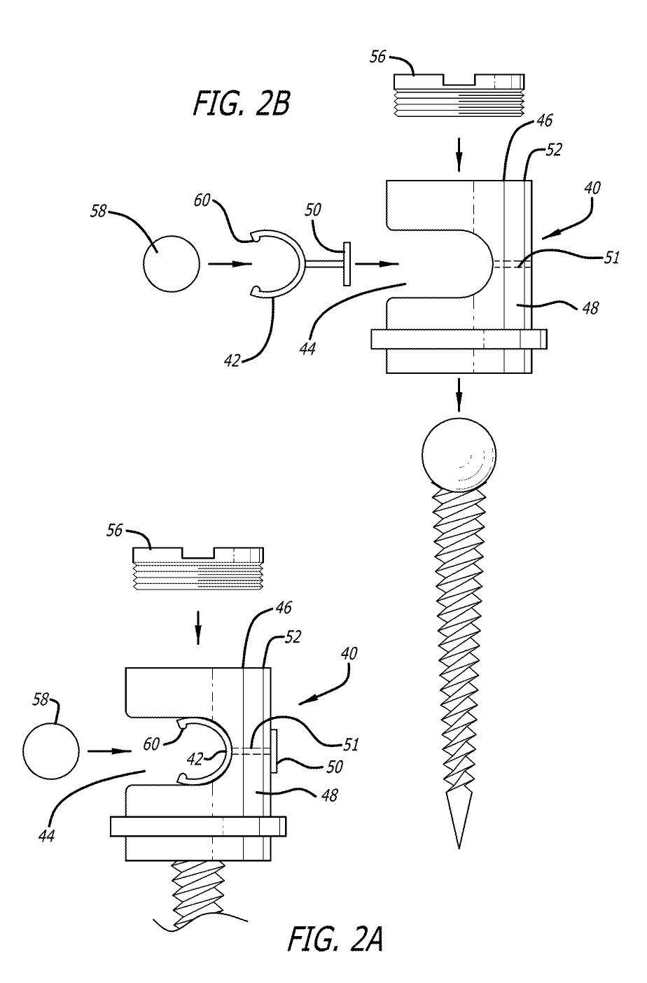

FIGS. 2a and 2b illustrate an alternative embodiment of the side-opening bone fastener assembly of the current invention that is generally configured like, and operates like the screw of FIG. 1. Specifically, fastener assembly 40 differs from that of

FIG. 1, in that the rod holder contacting member 42 is made to be inserted through the side opening 44 and not the top opening 46. Specifically, in the embodiment shown in these figures the rod holder contacting member 42 is in the form of a “U” shaped saddle that is connected within the connecting member 48 through the side opening 44. Although the rod holder contacting member 42 may be retained within the connecting member 48 through any suitable means, in the embodiment shown in FIG. 2, it is retained in the connecting member via a retaining member 50 that operates with a cooperative opening 51 in the connecting member 48, i.e., such as through a key-hole mechanism.

Although one embodiment of such a key-hole mechanism wherein the connecting member passes through the connecting member to engage the connecting member externally is shown in FIG. 2, it should be understood that the rod holder contacting member may be fixedly interconnected with the connecting member through any suitable means. For example, the rod holder contacting member may be molded or formed with a key hole mechanism or similar attachment means that are internal to the connecting member.

As in the previous embodiment, in the embodiment of the invention shown in FIG. 2, a screw thread 52 spirals from an upper opening 46 distally into the cylindrical body of the connecting member 48. A locking nut 56 engages the thread 52 in the upper opening of the connecting member 48 to force the arms of contacting member 42 down onto the rod 58. Although the locking nut 56 engages the rod 58 between the arms of the contacting member to prevent the rod from slipping from between the armatures, because the armatures are aligned parallel, absent some inner retaining means it would still be possible for the rod to move along the arms prior to full compression. Accordingly, some inner retaining means is desirable to ensure stable fixation of the rod within the contacting member of the connecting member prior to full compression. One exemplary alternative is shown in FIGS. 2a and 2b, in which the contacting member 42 comprises divots 60 formed into the upper and lower arms of the contacting member. It should be understood that while the embodiment shown in FIG. 2 shows divots formed in both the upper and lower arms, that a single larger divot could be formed on only one of the arms to serve the same purpose.

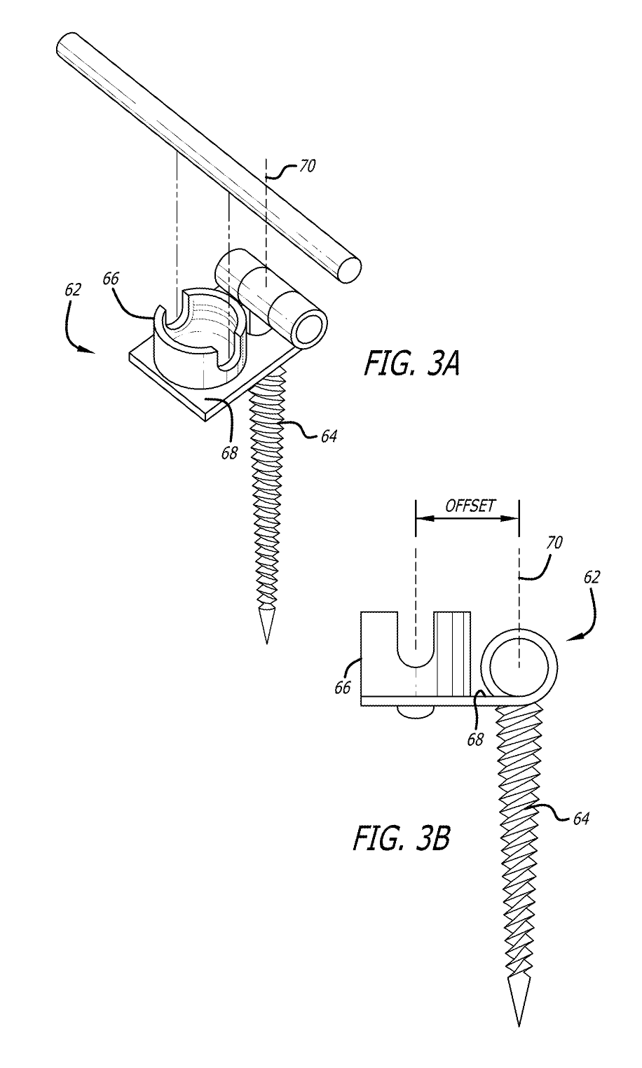

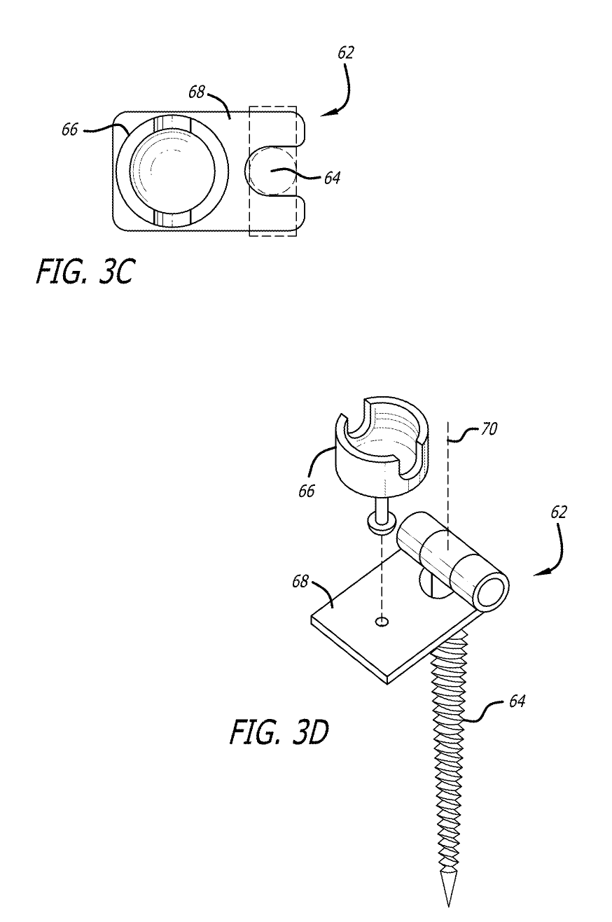

Finally, although all of the embodiments of the offset bone fastener system shown thus far have the rod being positioned in line with the axis of the screw, it should be understood that the side-opening connecting member can also be incorporated with an offset linkage in the form of an armature 62 (FIG. 3a) linked to the screw 64 and rod holder 66. As best seen in FIG. 3a, the offset linkage 68 is, in one embodiment, able to pivot azimuthally in a vertical plane about the bone fastener such that the side of the bone fastener on which the offset of the connecting member 66 is disposed, and the angle between the connecting member 66 and the longitudinal axis 70 of the screw 64 may be altered. In addition to the hinged offset linkage 68, which allows the angle of the connecting member 66 to be varied azimuthally in relation to the longitudinal axis 70 of the screw 64.

Although, one possible axial offset armature is shown in FIG. 3, above, it should be understood that a number of alternative arrangements may be used. First, with regard to the exemplary embodiment shown in FIGS. 3a to 3d, in this first embodiment the distance of the offset is fixed by the length of the offset linkage. In addition, in this first embodiment the connecting member is rotatably connected to the offset linkage to provide yet another degree of freedom in positioning the rod in relation to the overall assembly. However, it should be understood that these “degrees of freedom” are not necessary to the invention. For example, the connecting member could be fixedly attached to the offset linkage and the offset linkage itself could be fixedly attached to the bone fastener. In addition, while this embodiment shows the offset being a fixed offset distance, that is the offset distance is predetermined by the length of the offset linkage, it should be understood that the current invention also contemplates variable offset systems. Such a variable offset system could be provided through a plurality of different interchangeable linkage lengths that might be supplied with the bone fastener system. In such an embodiment the practitioner would determine the desired length, attach the correct offset linkage to the screw when placing the fastener into the patient. Alternatively, the fastener system of the current invention may include an adjustable offset linkage. In such an embodiment, the offset linkage may be provided with a plurality of connecting member openings arranged along the offset linkage. Such an arrangement would allow for the offset of the rod to be adjusted inward or outward transverse to the longitudinal axis of the screw by positioning the connecting member at desired positions along the linkage. Using such a system it is possible to provide an adjustable offset.

In addition to the above-variations, the bone fastener system of in accordance with the current invention may also include to facilitate osseous integration, to reduce the risk of infection in immunologically suppressed patients and/or for utilization in patients who have infection, but who require stabilization, and to permit the use of dissimilar metals and avoid the galvanic corrosive reactions. Examples of each of these different coatings is provided below, but for a more thorough discussion see U.S. Patent Publication No. 2008-0306554 A1, the disclosure of which is incorporated herein by reference.

Exemplary embodiments of coatings that facilitate osseous integration include, osteoconductive and osteoinductive substances, such as, for example, titanium oxide ceramic coatings, hydroxyapatite porous coatings, calcium phosphate, fluorine, and other surface treatments. Exemplary embodiments of coatings that may be used to reduce the risk of infection include, antimicrobial, antibiotic or antiseptic coatings, such as, for example, amoxicillin, cephamandol, carbenicillin, cephalothin, gentamicin, vancomycin, tobramycin, vancomycin incorporated into polymeric coatings such as, for example, poly lactic acid (PLA), poly-L-lactide (PLLA), poly-D-L-lactide (PDLLA), polyglycolic acid (PGA), phosphate PLA, and proteolactide coglycolide (PLGA). Alternatively, metal ion coatings, such as, silver or zinc ions, or fibrinolytic agents, such as, for example, heparin or fibronectin may be incorporated into the fastener systems of the current invention. Finally, exemplary embodiments of galvanic coatings include, parylene type coatings, such as, for example, Parylene N, Parylene C, Parylene D, and Parylene HI.

Although the above has only focused on the construction of the bone fastener assembly and system of the current invention, it should be understood that the invention is also directed to a method of stabilizing a bone using the system described herein. For example using the embodiment shown in FIG. 1, during surgery, the screw 14 is driven into a bone, e.g., a pedicle, at a desired angle. The rod holder 16 is swiveled relative to the screw 14 to a desired orientation to align the transverse passageway of the rod holder 16 with a desired rod orientation. The rod 12 is then placed into the rod holder 16 and secured by a screw into the threaded cylindrical body 28 to press the rod 12 and rod holder 16 together to lock the construct in the desired position. Referring to FIG. 3, in one embodiment, once the screw 14 and linkage offset 18 are positioned the offset of the rod holder 16 is adjusted and the rod 12 is inserted into the rod holder 16 is locked into the desired position.

Regardless of the specific embodiment, the screw 14, the connecting member/rod holder 16 and (optionally) offset linkage 18 may be assembled intraoperatively by the surgical team or they may be preassembled. Preferably, the components are preassembled and locked in place to prevent disassembly in order to simplify their use in surgery.

While the specific embodiments used to illustrate the invention show the bone fastener assembly in the form of a pedicle screw useful to attach a rod to a vertebrae, the bone fastener assembly may connect to a bone in any of the variety of ways known in the art and may be utilized in any of the variety of constructs known in the art to stabilize bones at any location within the body.

Although examples of a bone fastener assembly and its use have been described and illustrated in detail, it is to be understood that the same is intended by way of illustration and example only and is not to be taken by way of limitation. The invention has been illustrated in the form of a pedicle screw assembly for use in assembling stabilizing constructs to connect vertebrae of the human spine. However, the bone fastener assembly may be configured with other kinds of fasteners and connecting members to assemble other kinds of constructs to stabilize bones and bone fragments at any location in the body. Accordingly, variations in and modifications to the bone fastener assembly and its use will be apparent to those of ordinary skill in the art. The various illustrative embodiments illustrate alternative configurations of various component parts such as screw seats, screws, connecting members, pivot mechanisms, swivel mechanisms, and inserts among others. In most cases, and as will be readily understood by one skilled in the art, the alternative configuration of a component part in one embodiment may be substituted for a similar component part in another embodiment. Likewise, the various mechanisms illustrated for attaching the screw seat to the rod holder may be interchanged. Furthermore, throughout the exemplary embodiments, where component part mating relationships are illustrated, the gender of the component parts may be reversed as is known in the art within the scope of the invention. The following claims are intended to cover all such modifications and equivalents

What is claimed is:

1. A side-opening bone fastener system for connecting a bone to a stabilizing member comprising:
a bone fastener having an elongated shank including a bone engaging portion at a first end, a head at an opposite, second end, and a longitudinal screw axis extending therebetween;
a connecting member interconnected with said bone fastener, said connecting member having a longitudinal connecting member axis extending in the direction of the longitudinal screw axis, and a rod receiving channel extending at least partially through the connecting member transverse to the longitudinal axis for receiving a spinal rod, the receiving channel having an opening formed in a lateral face of the connection member along the axis of the stabilizing member but laterally offset therefrom such that the stabilizing member may be placed into the receiving channel of the connecting member laterally from the side of said connecting member.

2. The side-opening bone fastener system of claim 1, wherein the head is elongated in a direction transverse to the longitudinal screw axis such that the bone fastener is generally “T”-shaped.

3. The side-opening bone fastener system of claim 1, wherein the connecting member is rotatably mounted to the bone fastener such that the connecting member rotates about at least one of the longitudinal axis of the connecting member and an axis transverse to the longitudinal connecting member axis.

4. The side-opening fastener system of claim 1, wherein the connecting member is rotatably mounted to the bone fastener such that the connecting member rotates about the longitudinal axis of the fastener in a plane horizontal to the longitudinal axis of the fastener.

5. The side-opening bone fastener system of claim 1, wherein the stabilizing member receiving channel extends fully through the connecting member.

6. The side-opening bone fastener system of claim 1, wherein the stabilizing member is an elongated rod.

7. The offset bone fastener system of claim 1, wherein the assembly is made of stainless steel.

8. The offset bone fastener system of claim 1, wherein the head of the bone fastener further comprises a recess disposed in the head thereof and designed to receive a screw driver from the group consisting of: flat-headed, phillips head, allen wrench, and square headed.

9. The offset bone fastener system of claim 1, wherein the screw further comprises a slotted groove disposed longitudinally along the tip and designed to allow the screw to be self-tapped.

10. The side-opening bone fastener system of claim 1, wherein the connecting member further comprising a threaded channel and a cooperatively threaded mating nut having a plurality of engaging teeth disposed on the lower surface thereof, said threaded channel and mating nut being disposed such that said nut frictionally locks the stabilizing member within the channel of the connecting member.

11. The side-opening bone fastener system of claim 10, wherein the connecting member further comprises a stabilizing member contact member disposed between the stabilizing member and the mating nut, such that the threaded mating nut urges a contact surface of the stabilizing member contact member against the stabilizing member to lock the stabilizing member within the channel of the connecting member.

12. The side-opening bone fastener system of claim 11, wherein the contact surface of the stabilizing member contact member is shaped to conform to the outer contour of the stabilizing member.

13. The side-opening bone fastener system of claim 11, wherein the contact surface of the stabilizing member contact member has at least one raised feature thereon to increase the frictional force exerted on the stabilizing member when the stabilizing member contact member is engaged thereagainst.

14. The side-opening bone fastener system of claim 11, wherein the stabilizing member contact member is disposed within the channel opposite the lateral opening in the connecting member.

15. The side-opening bone fastener system of claim 14, wherein the stabilizing member contact member is an elongated U-shaped member, wherein the opening in the U-shaped member runs along an axis parallel to the lateral channel opening in the connecting member.

16. The side-opening bone fastener system of claim 15, wherein stabilizing member contact member further comprises a retaining member that securely interconnects the stabilizing member contact member to the connecting member.

17. The side-opening bone fastener system of claim 16, wherein the retaining member interconnects with the connecting member via a key-hole mechanism.

18. The side-opening bone fastener system of claim 14, wherein the stabilizing member contact member has a contact surface for engaging the stabilizing member, and wherein said contact surface has at least one raised feature thereon to increase the frictional force exerted on the stabilizing member when the stabilizing member contact member is engaged thereagainst.

19. The side-opening bone fastener system of claim 1, further comprising an offset linkage interconnecting said connecting member and the head of the bone fastener, wherein the connecting member is disposed on the offset linkage such that the longitudinal connecting member axis is laterally offset from the longitudinal screw axis by a predefined distance.

20. The side-opening bone fastener system of claim 19, wherein the offset linkage is pivotally interconnected with said head such that the connecting member azimuthally pivots about the head of said bone fastener; and wherein the connecting member is rotatably mounted to the offset linkage such that the connecting member rotates about the longitudinal connecting member axis.

21. The side-opening bone fastener system of claim 19, wherein the connecting member and the offset linkage are a single integrated piece.

22. The side-opening bone fastener system of claim 19, wherein the lateral offset distance between the longitudinal screw axis and longitudinal connecting member axis is fixed.

23. The side-opening bone fastener system of claim 19, wherein the lateral offset distance between the longitudinal screw axis and longitudinal connecting member axis is variable.

24. The side-opening bone fastener system of claim 19, wherein the system comprises a plurality of interchangeably removable offset linkages each having a different predefined fixed offset distance.

25. A vertebral alignment/fixation method comprising utilizing a plurality of the side-opening bone fastener systems as described in claim 1 to align at least one vertebral body.
